(19) United States
(12) Patent Application Publication
(10) Pub. No.: US 2012/0021057 A1
(43) Pub. Date: Jan. 26, 2012

(54) ONCE-DAILY ORAL IR/CR PRAMIPEXOLE FORMULATION

(75) Inventor: Thinnayam Naganathan Krishnamurthy, Scarborough (CA)

(73) Assignee: Purdue Pharma, Ontario (CA)

(21) Appl. No.: 13/259,588

(22) PCT Filed: Apr. 7, 2010
(86) PCT No.: PCT/CA10/00482

§ 371 Date: Sep. 23, 2011

(30) Foreign Application Priority Data
Apr. 9, 2009 (CA) PCT/CA2010/000482

(51) Int. Cl. A61K 31/428; A61K 9/50; A61P 25/00; A61K 9/14; A61P 25/16

(52) U.S. Cl. 424/490; 514/367; 427/2.21

(57) ABSTRACT

An oral once-daily pramipexole formulation, comprising an immediate-release component and a controlled-release component, is provided wherein in preferred embodiments, both the immediate-release component and the controlled-release component comprise pramipexole The formulation is preferably in the form of a coated bead A method of manufacturing said formulation is also provided.

### FIELD OF THE INVENTION

The present invention relates generally to dopamine agonists. More particularly, the present invention relates to once-daily immediate- and controlled-release pramipexole formulations.

### BACKGROUND OF THE INVENTION

Pramipexole is a nonergot dopamine agonist, with particular high affinity for the D3 receptor subtype. It is most often indicated in treating Parkinson's Disease (PD) and restless legs syndrome (RLS), where it likely acts to stimulate dopamine receptors in the striatum, thereby restoring the dopamine signals needed for proper functioning of the basal ganglia.

Prescription pramipexole is typically available as a dihydrochloride (under the trademark Mirapex®) for administration as an oral thrice-daily dosage form (for the treatment of PD) or once-daily at bedtime for RLS. The drug is generally well tolerated and can be taken with or without food.

There is a need for a once-daily pramipexole dosage form that can treat or prevent PD and/or RLS during the daytime hours, over the course of a 24 hour period. A once-daily dosage form would improve patient compliance. It would also provide a sustained release of drug over a 24 hour period.

Pramipexole is known to be highly unstable, particularly in the presence of excipients therewith. Excessive heat and humidity can degrade pramipexole and reduce its efficacy over time, thus reducing the shelf-life of pramipexole dosage forms. Thus, there is a need for a pramipexole dosage form that reduces or eliminates the effects of heat and moisture, and prolongs shelf-life, thereby prolonging efficacy of the formulation.

Patients with PD, especially those with advanced PD, have undergone percutaneous endoscopic gastrostomy or jejunostomy (PEG or PEJ), are intubated, or have otherwise undergone invasive procedures, may have difficulty swallowing medication. Current pramipexole dosage forms are often not tolerable for certain patients with PD, or present considerable difficulty for ingesting the medication. A once-daily pramipexole dosage form which is suited for PD (or other) patients who have difficulty ingesting currently available pramipexole medicaments, would be particularly beneficial.

It is, therefore, desirable to provide a once-daily pramipexole formulation which is more resistant to degradation, and more practical for advanced PD sufferers.

### SUMMARY OF THE INVENTION

It is an object of the present invention to obviate or mitigate at least one disadvantage of previous once-daily pramipexole formulations.

In a first aspect, the present invention provides an oral once-daily pramipexole formulation, comprising an immediate-release component and a controlled-release component. The immediate-release component and controlled-release component comprise pramipexole.

In one embodiment, the formulation is a coated bead. The immediate-release component and controlled-release component form layers on the coated bead.

The formulation can comprise one or more excipients.

Further, the formulation can comprise a moisture barrier coating. Ideally, the formulation is cured.

Ideally, at least 20% of the dosage of pramipexole is released in vitro within 2 hours after administration, and at least 40% of the dosage of pramipexole is released in vitro within 4 hours after administration.

Advantageously, the formulation is in a form for admixture with food for administering to a subject in need thereof.

In another aspect of the present invention, there is provided a method of producing an oral once-daily pramipexole formulation having both immediate-release and controlled-release layers, comprising the steps of: a) applying a pramipexole solution to coat a pharmaceutically-acceptable bead; b) applying a coating to the pramipexole-coated bead to produce a bead having a controlled-release layer; c) applying a film coating to the bead produced in step b); d) curing the bead produced in step c); and e) applying a second pramipexole solution to coat the bead produced in step d) to produce a bead having the controlled-release layer and an immediate-release layer. The method can also comprise the step of applying a moisture barrier coat on the bead.

In yet another aspect of the present invention there is provided a method of treating or preventing a disease in a subject in need thereof comprising administering the pramipexole formulation to said subject. The disease is typically one which is associated with a dopamine receptor, such as PD or RLS, for example.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 shows a typical flowchart of the manufacture of a once-daily IR/CR pramipexole formulation in accordance with the present invention.

FIG. 2 shows dissolution profiles of pramipexole formulations.

FIG. 3 shows the results of a single dose PK study under fasting conditions.

FIG. 4 shows the results of a comparative stability study of pramipexole CR beads in varying conditions over three months.

FIG. 5 shows the results of a comparative stability study, similar to that shown in FIG. 4, of pramipexole CR beads in varying conditions over three months.

FIG. 6 shows the results of a pharmacokinetic study.

FIG. 7 shows the results of a steady-state PK.

FIG. 8 shows the results of a PK study.

### DETAILED DESCRIPTION

Generally, the present invention provides a once-daily prampipexole formulation. In particular, the present invention provides an oral once-daily pramipexole formulation, comprising an immediate-release (IR) component and a controlled-release (CR) component. The immediate-release component and controlled-release component comprise pramipexole and/or one or more suitable excipients. In one embodiment, the formulation is a coated bead, which can be cured. The immediate-release component and controlled-release component typically form layers on the coated bead. Further, the formulation can comprise a moisture barrier coating. Ideally, at least 20% of the dosage of pramipexole is released in vitro within 2 hours after administration, and at least 40% of the dosage of pramipexole is released in vitro within 4 hours after administration. The formulation can be in a form for admixture with food for administering to a subject in need thereof.

In accordance with another aspect of the present invention, there is provided a method of producing a once-daily oral pramipexole formulation having both immediate-release and controlled-release layers, comprising the steps of: a) applying a pramipexole solution to coat a pharmaceutically-acceptable bead; b) applying a coating to the pramipexole-coated bead to produce a bead having a controlled-release layer; c) applying a film coating to the bead produced in step b); d) curing the bead produced in step c); and e) applying a second pramipexole solution to coat the bead produced in step d) to produce a bead having the controlled-release layer and an immediate-release layer.

The oral pramipexole formulation as described herein can be any pharmaceutically- and/or therapeutically-acceptable dosage form for administration to a subject in need thereof. In one exemplary embodiment, the formulation is a capsule comprising the IR/CR coated beads as described herein. In another embodiment, the formulation can be in the form of coated beads which are added to soft food. This embodiment may be particularly useful for subjects who have difficulty ingesting oral dosage forms, such as capsules or tablets.

As used herein, a “pramipexole solution” can include any solution comprising a pharmaceutically- and/or therapeutically-acceptable amount of pramipexole, salt thereof, or conjugate thereof. Typically, the pramipexole is dissolved in water. A binder (eg. Opadry®) and/or an anti-sticking agent (e.g. silicon dioxide) can be added to the solution. Any suitable buffer, carrier or excipient can be added to the pramipexole solution as needed.

The core beads onto which the pramipexole solution is applied can be any suitable beads in the art, such as microcrystalline cellulose (e.g., Avicel®), sugar beads, or the like. The pramipexole solution is applied to the beads to provide a uniform immediate-release core layer of active ingredient. Typically, the solution is sprayed onto the beads using an appropriate spray-coat device.

As used in the present application, “controlled-release” means that release of the active ingredient, i.e., pramipexole, is gradually and predictably controlled over a particular time period. Ideally, a pramipexole formulation in accordance with the present invention will provide a controlled-release of pramipexole over a 24-hour period. This provides a formulation which can be administered once-daily. Thus, the controlled-release layer as used herein is one which incorporates a coating material that allows for controlled release of pramipexole in a layer thereunder.

Optimally, the controlled-release layer coated bead is cured to stabilize the dissolution profile of the completed dosage form during its shelf life. Curing can be done using any suitable means and temperature, for any suitable duration of time.

The outer immediate-release layer of pramipexole can be the same as or different to the immediate-release core layer, i.e., can comprise any or all of the excipients carriers, buffers, in the same or different amounts. Of the total amount of pramipexole used in the present dosage form, a percentage is used in the outer immediate-release layer such that more than 20% of the drug is released within the first 2 hours and 40% is released within 4 hours after administration of the dosage form. The dosage form is flexible such that various dosage strengths can be derived (for example, 0.125 mg to 4.5 mg) from a common bead formation.

Optionally, the outer immediate-release layer can be coated with a moisture barrier coating. Any suitable moisture barrier coating can be used. Ideally, the moisture barrier coating is one which maintains the stability of the pramipexole formulation and/or prolongs the “shelf life” of the formulation. In other words, the moisture barrier coating ideally prevents any adverse degradation due to excessive exposure to environmental conditions, such as, for example, high humidity. Suitable excipients, in combination with the moisture barrier coating, can also be used to promote the integrity of the formulation. As would be known in the art, pramipexole has a tendency to degrade at high levels of humidity, particularly in the presence of excipients.

The immediate- and controlled-release layered bead formulation can then be encapsulated in any suitable manner known in the art. However, the layered bead can also be used an additive used in admixture with food for administering to a subject in need thereof, particularly those subjects who have difficulty swallowing typical pramipexole dosage forms. As one example of an additive, the coated bead can be sprinkled on soft food prior to ingestion.

### EXAMPLES

### Example 1

### Pramipexole Formulation

One embodiment of a pramipexole formulation in accordance with the present invention is summarized in Table 1.

TABLE 1

| | | Strength (label claim): |
|---|---|---|
| | | 0.75 mg/capsule |
| | | Formula F |
| Component and | | Quantity |
| Quality Standard | | per unit |
| (and Grade, if applicable) | Function | (mg) |
| Pramipexole dihydrochloride | active | 0.75 |
| monohydrate, HS | | |
| Sugar spheres | substrate | — |
| Microcrystalline cellulose | substrate | 77.14 |
| Opadry clear YS-1-7006 | binder | 3.10 |
| Silicon dioxide | anti-sticking | 3.93 |
| | agent | |
| Kollicoat SR 30 D solids | control coat | 12.98 |
| Kollicoat IR | control coat | 0.71 |
| Propylene glycol | plasticizer | 1.31 |
| Talc | glidant | 10.36 |
| Polysorbate 80 | surfactant | 0.02 |
| Opadry AMB white | moisture barrier | 8.81 |
| | coat | |
| Purified water | solvent | q.s. |

### Example 2

### Method of Manufacturing an IR/CR Pramipexole Formulation

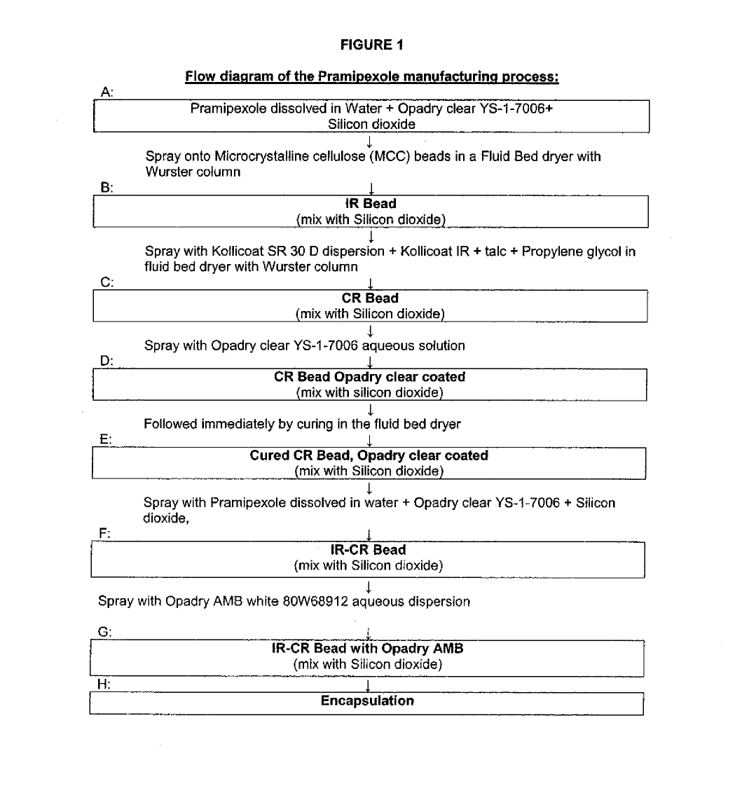

FIG. 1 shows a typical flowchart of the manufacture of a once-daily IR/CR pramipexole bead formulation.

At step A, a pramipexole solution is produced from pramipexole dissolved in water, Opadry Clear® YS-1-7006 and silicon dioxide.

At step B, the pramipexole solution is sprayed onto microcrystalline cellulose (MCC) beads in a fluid bed dryer with a Wurster column. This produces an immediate-release (IR) bead.

At step C, the IR bead is mixed with silicon dioxide and sprayed with Kollicoat SR 30 D dispersion, Kollicoat IR, talc, and propylene glycol in a fluid bed dryer with a Wurster column. A controlled-release (CR) bead is thus formed.

At step D, the CR bead is mixed with silicon dioxide and sprayed with Opadry Clear® YS-1-7006 aqueous solution. Opadry protects the beads from agglomeration during the curing step E (see below).

Immediately following step D, the clear-coated CR bead is mixed with silicon dioxide and cured for 6 hours in the fluid bed dryer at 60° C. (step E). Surprisingly, the curing was found to be necessary to stabilize the dissolution and assay during the shelf-life of pramipexole CR beads.

At step F, the cured CR bead is mixed with silicon dioxide and sprayed with pramipexole dissolved in water, Opadry Clear YS-1-7006 and silicon dioxide, to produce an IR/CR bead.

At step G, the IR/CR bead is mixed with silicon dioxide and sprayed with Opadry AMB white aqueous dispersion. This provides a moisture barrier coat to the IR/CR pramipexole bead. The moisture barrier coat (Opadry AMB) is important for protecting the beads from moisture, thus preventing degradation and reducing impurity levels during the entire shelf-life of the product. The coating is especially useful even at high heat/humidity (30° C./65% relative humidity) storage condition.

Finally, the moisture barrier coated IR/CR bead is mixed with silicon dioxide and encapsulated (step H) to produce an IR/CR pramipexole dosage form.

### Example 3

### In Vitro Pramipexole Dissolution Profile

Table 2 shows the results of an in vitro dissolution study of the pramipexole formulation in accordance with the present invention. The dissolution study was conducted using USP paddle method at 100 rpm speed with 500 ml simulated gastric fluid without enzyme as the dissolution medium and measuring the % dissolved by HPLC method using UV detector at 260 nm.

TABLE 2

| | | (b) Results |
|---|---|---|
| | | % Pramipexole Dihydrochloride |
| Tests | (a) Specification | Released (Range) |
| Dissolution | Time Hours | % Released |
| USP paddle method, | 1 | 20 (20-21) |
| 100 rpm at 37° C. | 2 | 30 (29-30) |
| Simulated Gastric | 4 | 46 (45-47) |
| Fluid, without | 6 | 58 (57-60) |
| enzyme pH 1.2 | 8 | 67 (66-68) |
| HPLC/UV | 10 | 73 (72-75) |
| at 260 nm | 12 | 77 (76-79) |
| | 16 | 84 (83-85) |
| | 18 | 87 (86-89) |
| | 20 | 89 (87-91) |
| | 24 | 92 (90-94) |

### Example 4

### Dissolution Profile of Once-Daily Pramipexole CR Beads

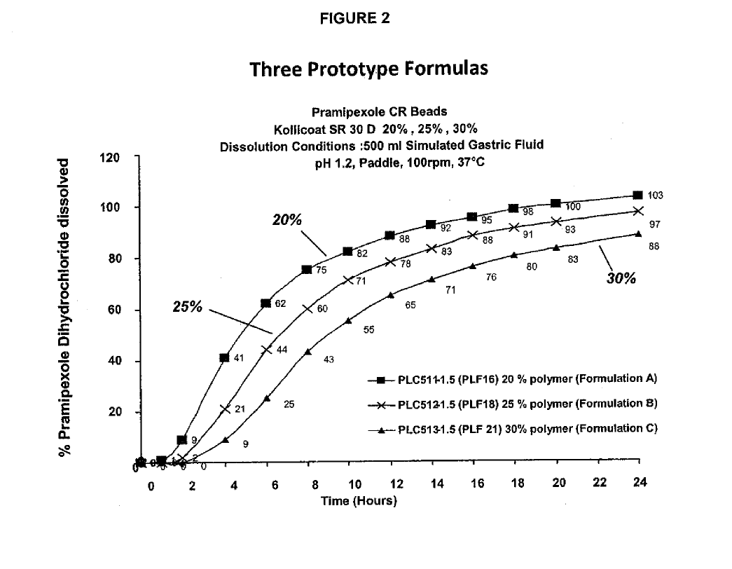

Three prototype formulations were developed, containing Kollicoat SR-30D (control coat) 20, 25, and 30% respectively as the rate controlling excipient using sugar beads as substrate. FIG. 2 shows the dissolution profiles of these formulations: 20% (squares), 25% (crosshairs) and 30% (triangles).

### Example 5

### Single Dose Pharmacokinetic Study of Pramipexole CR Capsules

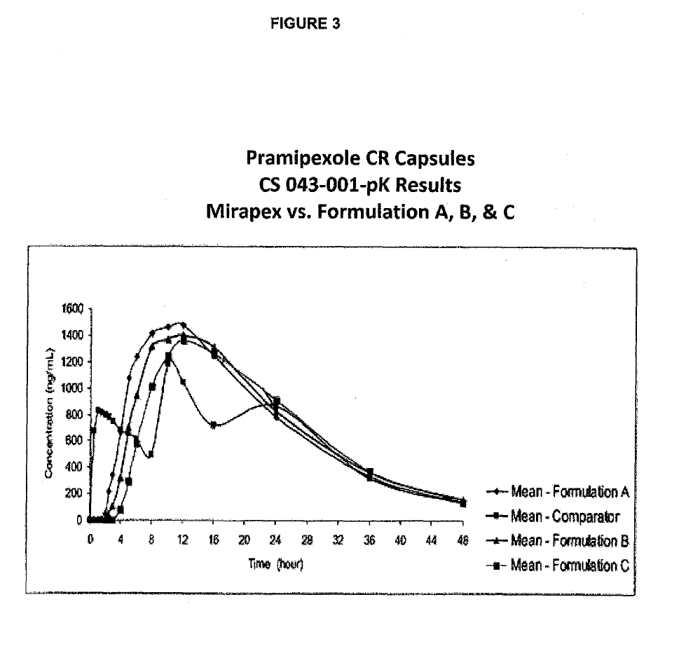

FIG. 3 shows the pharmacokinetic results of a single dose PK study conducted under fasted conditions. The study compared a comparator—IR Pramipexole (Mirapex 0.5 mg dosed every 8 hours) against three prototype formulations (Formulation A—20% Kollicoat SR polymer, Formulation B—25% Kollicoat SR and Formulation C—30% Kollicoat SR) dosed once as 1.5 mg. The results show that all 3 formulations were bioequivalent to the IR pramipexole; however, a lag time of 2-3 hours was observed and the Cmax was slightly higher than the desired profile. A summary of the PK results is shown in Table 3 and the data points of FIG. 3 are shown in Tables 3A-D.

### Example 6

### Stability Study of Pramipexole CR Beads

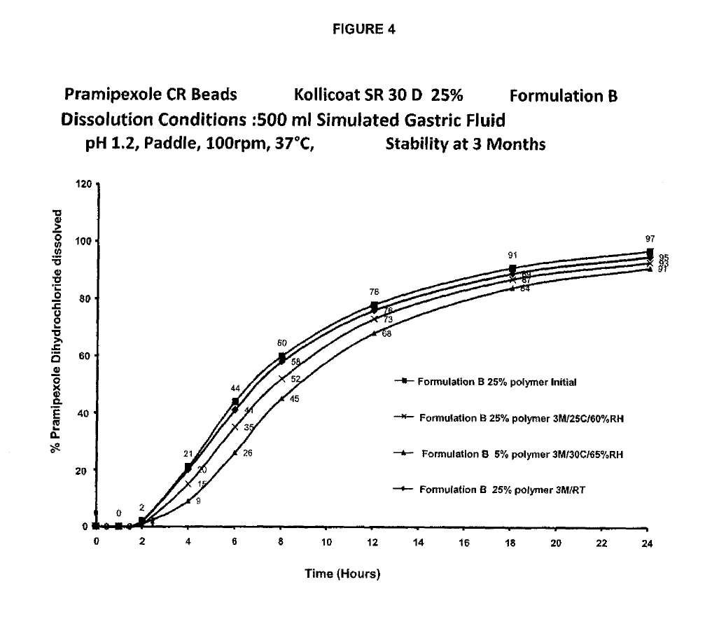

FIG. 4 shows the results of a comparative stability study of a pramipexole CR bead Formulation B at ICH stability storage conditions over three months. The beads were coated with Kollicoat SR 30D—25%. Though the CR capsules were stable at ambient room-temperature, the dissolution profile showed a significant reduction (10-15%) when stored at 25° C. and 30° C.

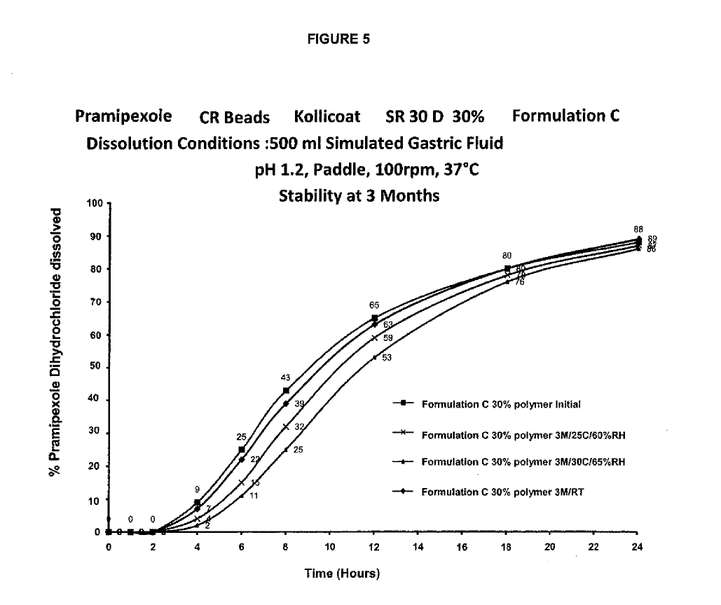

A similar study was performed on Formulation C—containing 30% Kollicoat SR 30D as the rate controlling excipient. The results are shown in FIG. 5.

TABLE 3
Pharmacokinetic results: Comparison of Pramipexole 1.5 mg CR capsules dosed
once/treatment (Formulations A, B & C) vs. Mirapex 0.5 mg dosed 3 times.

| | | | | | |
|---|---|---|---|---|---|
| Tmax | Cmax | T_{1/2z} | λ_{z} | AUC_{T} | AUC_{inf.} |
| (h) | (ng/mL) | (h) | (h⁻¹) | (ng · h/mL) | (ng · h/mL) |

Formulation A: Purdue Pharma Pramipexole 1.5 mg CR capsule.

| | | | | | | |
|---|---|---|---|---|---|---|
| Mean | 11.0 | 1552.5 | 9.5 | 0.1 | 34229.0 | 36471.3 |
| SD | 2.5 | 296.9 | 2.1 | 0.0 | 6304.6 | 7480.4 |
| Min. | 8.0 | 1160.0 | 6.5 | 0.0 | 257776.3 | 27190.6 |
| Max | 16.0 | 2110.0 | 14.1 | 0.1 | 43699.7 | 48170.5 |

Formulation B: Purdue Pharma Pramipexole 1.5 mg CR capsule.

| | | | | | | |
|---|---|---|---|---|---|---|
| Mean | 12.0 | 1488.0 | 9.9 | 0.1 | 33512.3 | 35973.0 |
| SD | 2.6 | 397.3 | 2.0 | 0.0 | 7708.9 | 8833.2 |
| Min. | 8.0 | 872.0 | 6.6 | 0.1 | 23464.4 | 24649.6 |
| Max | 16.0 | 2040.0 | 13.7 | 0.1 | 47410.7 | 54164.3 |

Formulation C: Purdue Pharma Pramipexole 1.5 mg CR capsule.

| | | | | | | |
|---|---|---|---|---|---|---|
| Mean | 12.0 | 1440.8 | 9.2 | 0.1 | 31525.1 | 34064.6 |
| SD | 2.5 | 273.3 | 1.8 | 0.0 | 5146.7 | 6259.5 |
| Min. | 8.0 | 1100.0 | 6.1 | 0.1 | 23741.0 | 24726.5 |
| Max | 16.0 | 1950.0 | 12.8 | 0.1 | 43345.0 | 49197.0 |

Comparator: Boehringer Ingelheim Mirapex ® 0.5 mg IR tablet.

| | Tmax | | Cmax | | | | | |
|---|---|---|---|---|---|---|---|---|
| | (h) | | (ng/mL) | | T_{1/2z} | λ_{z} | AUC_{T} | AUC_{inf.} |
| | Tab 1 | Tab 2 | Tab 1 | Tab 2 | (h) | (h⁻¹) | (ng · h/mL) | (ng · h/mL) |
| Mean | 1.0 | 10.0 | 968.8 | 1198.2 | 8.7 | 0.1 | 28357.0 | 30061.2 |
| SD | 0.8 | 0.8 | 198.8 | 172.3 | 1.7 | 0.0 | 3367.4 | 4370.3 |
| Min. | 0.5 | 10.0 | 713.0 | 978.0 | 6.8 | 0.1 | 22995.5 | 23598.2 |
| Max | 3.0 | 12.0 | 1360.0 | 1520.0 | 12.0 | 0.1 | 34231.5 | 38359.1 |

TABLE 3A
Pramipexole plasma concentrations (pg/mL) - Formulation A

| Formulation A: | | Sampling time (hour) | | | | | | | | | |
|---|---|---|---|---|---|---|---|---|---|---|---|
| Vol. ID. | Period | 0 | 0.5 | 1 | 1.5 | 2 | 2.5 | 3 | 4 | 5 | 6 |
| PELAL 001 | Jul. 30, 2005 | 0 | 0 | 0 | 0 | 83.5 | 356 | 433 | 1030 | 1400 | 1500 |
| PENMA 002 | Jul. 23, 2005 | 0 | 0 | 0 | 0 | 82.5 | 186 | 279 | 572 | 1110 | 1140 |
| BRAEN 003 | Aug. 13, 2005 | 0 | 0 | 0 | 0 | 0 | 153 | 414 | 604 | 986 | 990 |
| FORMA 005 | Aug. 06, 2005 | 0 | 0 | 0 | 0 | 83.4 | 231 | 402 | 736 | 1090 | 1350 |
| LATLU 006 | Jul. 30, 2005 | 0 | 0 | 0 | 0 | 0 | 204 | 263 | 516 | 1370 | 1380 |
| JUBCL 007 | Aug. 13, 2005 | 0 | 0 | 0 | 0 | 77.1 | 235 | 340 | 606 | 1140 | 1050 |
| ROYER 008 | Jul. 23, 2005 | 0 | 0 | 0 | 0 | 0 | 128 | 235 | 589 | 1000 | 1180 |
| COUPA 009 | Jul. 23, 2005 | 0 | 0 | 0 | 0 | 0 | 179 | 212 | 378 | 936 | 1340 |
| DOIMA 010 | Jul. 30, 2005 | 0 | 0 | 0 | 0 | 119 | 347 | 499 | 1030 | 756 | 1260 |
| CARDO 011 | Aug. 06, 2005 | 0 | 0 | 0 | 0 | 82.4 | 230 | 477 | 832 | 1230 | 1500 |
| ROSMI 012 | Aug. 13, 2005 | 0 | 0 | 0 | 0 | 0 | 122 | 222 | 436 | 825 | 890 |
| MENJO 013 | Aug. 13, 2005 | 0 | 0 | 0 | 0 | 50.8 | 163 | 306 | 622 | 930 | 1170 |

| | | | | | | | | | | |
|---|---|---|---|---|---|---|---|---|---|---|
| MEAN | 0.0 | 0.0 | 0.0 | 0.0 | 48.2 | 211.2 | 340.2 | 662.6 | 1064.4 | 1229.2 |
| STD | 0.0 | 0.0 | 0.0 | 0.0 | 45.0 | 75.7 | 101.9 | 208.8 | 199.8 | 194.4 |
| CV | 0.0 | 0.0 | 0.0 | 0.0 | 93.4 | 35.8 | 29.9 | 31.5 | 18.8 | 15.8 |

| Formulation A: | | Sampling time (hour) | | | | | | |
|---|---|---|---|---|---|---|---|---|
| Vol. ID. | Period | 8 | 10 | 12 | 16 | 24 | 36 | 48 |
| PELAL 001 | Jul. 30, 2005 | 1660 | 1740 | 1660 | 1280 | 1000 | 350 | 126 |
| PENMA 002 | Jul. 23, 2005 | 1300 | 1220 | 1160 | 871 | 538 | 226 | 102 |
| BRAEN 003 | Aug. 13, 2005 | 978 | 1070 | 1160 | 1040 | 691 | 286 | 117 |
| FORMA 005 | Aug. 06, 2005 | 1390 | 1570 | 1610 | 1670 | 1100 | 502 | 266 |
| LATLU 006 | Jul. 30, 2005 | 1460 | 1300 | 1310 | 1170 | 663 | 234 | 85.9 |
| JUBCL 007 | Aug. 13, 2005 | 1200 | 1150 | 1040 | 871 | 567 | 229 | 96.7 |
| ROYER 008 | Jul. 23, 2005 | 1330 | 1510 | 1590 | 1530 | 995 | 502 | 221 |
| COUPA 009 | Jul. 23, 2005 | 1700 | 1760 | 1920 | 1370 | 628 | 320 | 85.8 |
| DOIMA 010 | Jul. 30, 2005 | 1690 | 1570 | 1570 | 1380 | 942 | 483 | 294 |
| CARDO 011 | Aug. 06, 2005 | 1830 | 2110 | 1970 | 1480 | 729 | 248 | LOQ |
| ROSMI 012 | Aug. 13, 2005 | 1050 | 1070 | 1230 | 989 | 697 | 282 | 98.4 |
| MENJO 013 | Aug. 13, 2005 | 1470 | 0520 | 1560 | 1560 | 896 | 258 | 68.4 |

| | | | | | | | |
|---|---|---|---|---|---|---|---|
| MEAN | 1421.5 | 1465.8 | 1481.7 | 1267.6 | 787.2 | 326.7 | 130.1 |
| STD | 267.2 | 316.8 | 301.4 | 276.0 | 189.3 | 108.4 | 86.0 |
| CV | 18.8 | 21.6 | 20.3 | 21.8 | 24.1 | 23.2 | 66.1 |

LOQ = 50 pg/mL. Any concentration below 50 pg/mL is reported as (BQL) except at time pre-dose and times before first observed concentration. However in the calculation of summary statistics a zero is used.

TABLE 3B
Pramipexole plasma concentrations (pg/mL) - Formulation B

| Formulation B: | | Sampling time (hour) | | | | | | | | | | | | | | | | |
|---|---|---|---|---|---|---|---|---|---|---|---|---|---|---|---|---|---|---|
| Vol. ID. | Period | 0 | 0.5 | 1 | 1.5 | 2 | 2.5 | 3 | 4 | 5 | 6 | 8 | 10 | 12 | 16 | 24 | 36 | 48 |
| PELAL | Aug. 06, | 0 | 0 | 0 | 0 | 0 | 115 | 163 | 571 | 932 | 1400 | 1990 | 2040 | 2040 | 1620 | 1100 | 422 | 168 |
| 001 | 2005 | | | | | | | | | | | | | | | | | |
| PENMA | Jul. 30, | 0 | 0 | 0 | 0 | 0 | 69.6 | 136 | 310 | 563 | 849 | 1390 | 1350 | 1570 | 1650 | 773 | 280 | 110 |
| 002 | 2005 | | | | | | | | | | | | | | | | | |
| BRAEN | Jul. 23, | 0 | 0 | 0 | 0 | 0 | 57.6 | 94.7 | 285 | 589 | 684 | 777 | 797 | 872 | 834 | 651 | 354 | 165 |
| 003 | 2005 | | | | | | | | | | | | | | | | | |
| FORMA | Aug. 13, | 0 | 0 | 0 | 0 | 0 | 0 | 54.0 | 212 | 405 | 631 | 1150 | 1380 | 1400 | 1390 | 994 | 517 | 270 |
| 005 | 2005 | | | | | | | | | | | | | | | | | |
| LATLU | Aug. 06, | 0 | 0 | 0 | 0 | 0 | 68.3 | 143 | 400 | 821 | 1370 | 1920 | 1620 | 1430 | 1100 | 557 | 228 | 107 |
| 006 | 2005 | | | | | | | | | | | | | | | | | |
| JUBCL | Jul. 23, | 0 | 0 | 0 | 0 | 0 | 54.7 | 99.0 | 330 | 586 | 736 | 880 | 900 | 944 | 837 | 630 | 256 | 92.8 |
| 007 | 2005 | | | | | | | | | | | | | | | | | |
| ROYER | Jul. 30, | 0 | 0 | 0 | 0 | 0 | 103 | 176 | 461 | 871 | 811 | 1100 | 1190 | 1310 | 1680 | 1120 | 475 | 187 |
| 008 | 2005 | | | | | | | | | | | | | | | | | |
| COUPA | Jul. 30, | 0 | 0 | 0 | 0 | 0 | 0 | 95.4 | 289 | 688 | 937 | 1370 | 1590 | 1530 | 1530 | 908 | 332 | 142 |
| 009 | 2005 | | | | | | | | | | | | | | | | | |
| DOIMA | Aug. 06, | 0 | 0 | 0 | 0 | 0 | 0 | 94.3 | 334 | 754 | 1120 | 1760 | 1870 | 1900 | 1640 | 1150 | 697 | 318 |
| 010 | 2005 | | | | | | | | | | | | | | | | | |
| CARDO | Aug. 13, | 0 | 0 | 0 | 0 | 0 | 0 | 72.9 | 154 | 1120 | 1420 | 1630 | 1640 | 1600 | 1210 | 653 | 176 | 53.0 |
| 011 | 2005 | | | | | | | | | | | | | | | | | |
| ROSMI | Jul. 23, | 0 | 0 | 0 | 0 | 0 | 0 | 65.6 | 180 | 443 | 533 | 863 | 883 | 1010 | 1040 | 753 | 337 | 150 |
| 012 | 2005 | | | | | | | | | | | | | | | | | |
| MENJO | Jul. 23, | 0 | 0 | 0 | 0 | 0 | 61.1 | 133 | 299 | 647 | 832 | 942 | 1140 | 1180 | 1110 | 676 | 271 | 124 |
| 013 | 2005 | | | | | | | | | | | | | | | | | |

| | | | | | | | | | | | | | | | | | |
|---|---|---|---|---|---|---|---|---|---|---|---|---|---|---|---|---|---|
| MEAN | 0.0 | 0.0 | 0.0 | 0.0 | 0.0 | 44.1 | 110.6 | 318.8 | 701.6 | 943.6 | 1314.3 | 1366.7 | 1398.8 | 1303.4 | 830.4 | 362.1 | 157.2 |
| STD | 0.0 | 0.0 | 0.0 | 0.0 | 0.0 | 42.7 | 39.0 | 117.2 | 208.3 | 311.0 | 428.9 | 399.4 | 362.0 | 320.4 | 213.9 | 145.5 | 74.3 |
| CV | 0.0 | 0.0 | 0.0 | 0.0 | 0.0 | 44.1 | 110.6 | 318.8 | 701.6 | 943.6 | 1314.3 | 1366.7 | 1398.8 | 1303.4 | 830.4 | 362.1 | 157.2 |

LOQ = 50 pg/mL. Any concentration below 50 pg/mL is reported as (BQL) except at time pre-dose and times before first observed concentration. However in the calculation of summary statistics a zero is used.

TABLE 3C
Pramipexole plasma concentrations (pg/mL) - Formulation C

| Formulation C: | | Sampling time (hour) | | | | | | | | | | | | | | | | |
|---|---|---|---|---|---|---|---|---|---|---|---|---|---|---|---|---|---|---|
| Vol. ID. | Period | 0 | 0.5 | 1 | 1.5 | 2 | 2.5 | 3 | 4 | 5 | 6 | 8 | 10 | 12 | 16 | 24 | 36 | 48 |
| PELAL 001 | Aug. 13, 2005 | 0 | 0 | 0 | 0 | 0 | 0 | 0 | 98.1 | 285 | 458 | 926 | 1110 | 1500 | 1370 | 1360 | 465 | 163 |
| PENMA 002 | Aug. 06, 2005 | 0 | 0 | 0 | 0 | 0 | 0 | 0 | 101 | 264 | 625 | 1240 | 1770 | 1950 | 1500 | 799 | 264 | 111 |
| BRAEN 003 | Jul. 30, 2005 | 0 | 0 | 0 | 0 | 0 | 0 | 0 | 87.1 | 302 | 444 | 669 | 878 | 1120 | 1310 | 1010 | 449 | 178 |
| FORMA 005 | Jul. 23, 2005 | 0 | 0 | 0 | 0 | 0 | 0 | 0 | 143 | 370 | 540 | 850 | 1220 | 1240 | 1080 | 830 | 384 | 172 |
| LATLU 006 | Aug. 13, 2005 | 0 | 0 | 0 | 0 | 0 | 0 | 0 | 141 | 479 | 1650 | 1710 | 1520 | 1310 | 939 | 588 | 243 | 80.5 |
| JUBCL 007 | Jul. 30, 2005 | 0 | 0 | 0 | 0 | 0 | 0 | 0 | 72.7 | 233 | 375 | 831 | 1070 | 1100 | 949 | 670 | 239 | 84.8 |
| ROYER 008 | Aug. 06, 2005 | 0 | 0 | 0 | 0 | 0 | 0 | 0 | 78.7 | 245 | 384 | 820 | 1050 | 1330 | 1240 | 1020 | 491 | Mi |
| COUPA 009 | Aug. 06, 2005 | 0 | 0 | 0 | 0 | 0 | 0 | 0 | 63.8 | 292 | 433 | 433 | 928 | 1230 | 1420 | 930 | 444 | 143 |
| DOIMA 010 | Aug. 13, 2005 | 0 | 0 | 0 | 0 | 0 | 0 | 0 | 103 | 375 | 882 | 1280 | 1570 | 1750 | 1630 | 1140 | 629 | 311 |
| CARDO 011 | Jul. 23, 2005 | 0 | 0 | 0 | 0 | 0 | 0 | 0 | 53.9 | 287 | 560 | 1280 | 1650 | 1620 | 1410 | 830 | 199 | 54.3 |
| ROSMI 012 | Jul. 30, 2005 | 0 | 0 | 0 | 0 | 0 | 0 | 0 | 0 | 159 | 244 | 495 | 968 | 972 | 1100 | 1020 | 435 | 179 |
| MENJO 013 | Jul. 30, 2005 | 0 | 0 | 0 | 0 | 0 | 0 | 0 | 0 | 208 | 368 | 1050 | 1230 | 1210 | 1130 | 793 | 247 | 86.2 |

| | | | | | | | | | | | | | | | | | |
|---|---|---|---|---|---|---|---|---|---|---|---|---|---|---|---|---|---|
| MEAN | 0.0 | 0.0 | 0.0 | 0.0 | 0.0 | 0.0 | 0.0 | 78.5 | 291.6 | 580.3 | 965.3 | 1247.0 | 1361.0 | 1256.5 | 915.8 | 374.1 | 171.2 |
| STD | 0.0 | 0.0 | 0.0 | 0.0 | 0.0 | 0.0 | 0.0 | 45.5 | 84.9 | 373.4 | 367.0 | 304.6 | 289.3 | 219.9 | 211.0 | 133.3 | 121.7 |
| CV | 0.0 | 0.0 | 0.0 | 0.0 | 0.0 | 0.0 | 0.0 | 58.0 | 29.1 | 64.3 | 38.0 | 24.4 | 21.3 | 17.5 | 23.0 | 35.6 | 71.1 |

LOQ = 50 pg/mL. Any concentration below 50 pg/mL is reported as (BQL) except at time pre-dose and times before first observed concentration. However in the calculation of summary statistics a zero is used.

TABLE 3D
Pramipexole plasma concentrations (pg/mL) - Formulation R (comparator)

| Formulation R: | | Sampling time (hour) | | | | | | | | |
|---|---|---|---|---|---|---|---|---|---|---|
| Vol. ID. | Period | 0 | 0.5 | 1 | 1.5 | 2 | 2.5 | 3 | 4 | 5 |
| PELAL 001 | Aug. 06, 2005 | 0 | 979 | 981 | 832 | 893 | 729 | 701 | 662 | 583 |
| PENMA 002 | Jul. 30, 2005 | 0 | 1360 | 1100 | 1100 | 954 | 897 | 804 | 716 | 624 |
| BRAEN 003 | Jul. 23, 2005 | 0 | 371 | 965 | 1070 | 952 | 876 | 852 | 683 | 693 |
| FORMA 005 | Aug. 13, 2005 | 0 | 580 | 624 | 675 | 748 | 697 | 745 | 604 | 600 |
| LATLU 006 | Aug. 06, 2005 | 0 | 840 | 1020 | 814 | 730 | 677 | 681 | 643 | 684 |
| JUBCL 007 | Jul. 23, 2005 | 0 | 231 | 400 | 355 | 471 | 707 | 780 | 692 | 623 |
| ROYER 008 | Jul. 30, 2005 | 0 | 222 | 713 | 702 | 558 | 582 | 516 | 597 | 689 |
| COUPA 009 | Jul. 30, 2005 | 0 | 654 | 766 | 994 | 1100 | 1150 | 925 | 928 | 863 |
| DOIMA 010 | Aug. 06, 2005 | 0 | 1110 | 1000 | 910 | 747 | 705 | 766 | 725 | 679 |
| CARDO 011 | Aug. 13, 2005 | 0 | 653 | 944 | 877 | 874 | 825 | 839 | 703 | 655 |
| ROSMI 012 | Jul. 23, 2005 | 0 | 438 | 567 | 621 | 703 | 720 | 614 | 571 | 539 |
| MENJO 013 | Jul. 23, 2005 | 0 | 834 | 1030 | 943 | 775 | 683 | 623 | 581 | 557 |

| | | | | | | | | | |
|---|---|---|---|---|---|---|---|---|---|
| MEAN | 0.0 | 689.3 | 842.5 | 821.1 | 792.9 | 770.7 | 737.2 | 675.4 | 649.1 |
| STD | 0.0 | 351.9 | 222.5 | 209.0 | 176.0 | 149.0 | 115.8 | 96.0 | 85.3 |
| CV | 0.0 | 51.0 | 26.4 | 25.5 | 22.2 | 19.3 | 15.7 | 14.2 | 13.1 |

| Formulation R: | | Sampling time (hour) | | | | | | | |
|---|---|---|---|---|---|---|---|---|---|
| Vol. ID. | Period | 6 | 8 | 10 | 12 | 16 | 24 | 36 | 48 |
| PELAL 001 | Aug. 06, 2005 | 575 | 440 | 1230 | 1050 | 722 | 808 | 262 | 86.4 |
| PENMA 002 | Jul. 30, 2005 | 581 | 517 | 819 | 1180 | 790 | 873 | 272 | 119 |
| BRAEN 003 | Jul. 23, 2005 | 575 | 468 | 1170 | 988 | 765 | 1070 | 364 | 155 |
| FORMA 005 | Aug. 13, 2005 | 558 | 494 | 1050 | 1000 | 846 | 965 | 434 | 216 |
| LATLU 006 | Aug. 06, 2005 | 654 | 512 | 1300 | 977 | 670 | 802 | 186 | 76.9 |
| JUBCL 007 | Jul. 23, 2005 | 533 | 431 | 1110 | 892 | 587 | 699 | 324 | 103 |
| ROYER 008 | Jul. 30, 2005 | 674 | 573 | 1010 | 1060 | 765 | 1000 | 382 | 214 |
| COUPA 009 | Jul. 30, 2005 | 765 | 573 | 1520 | 1150 | 701 | 622 | 278 | 67.6 |
| DOIMA 010 | Aug. 06, 2005 | 674 | 504 | 1380 | 1140 | 834 | 989 | 454 | 246 |
| CARDO 011 | Aug. 13, 2005 | 602 | 486 | 1400 | 1140 | 653 | 918 | 248 | 80.6 |
| ROSMI 012 | Jul. 23, 2005 | 548 | 470 | 978 | 906 | 611 | 749 | 304 | 109 |
| MENJO 013 | Jul. 23, 2005 | 501 | 459 | 1000 | 855 | 574 | 709 | 177 | 64.2 |

| | | | | | | | | |
|---|---|---|---|---|---|---|---|---|
| MEAN | 603.3 | 493.9 | 1163.9 | 1028.2 | 709.8 | 850.3 | 307.1 | 128.1 |
| STD | 74.7 | 45.6 | 207.2 | 109.9 | 92.8 | 140.6 | 88.4 | 64.1 |
| CV | 12.4 | 9.2 | 17.8 | 10.7 | 13.1 | 16.5 | 28.8 | 50.0 |

LOQ = 50 pg/mL. Any concentration below 50 pg/mL is reported as (BQL) except at time pre-dose and time before first observed concentration. However in the calculation of summary statistics a zero is used.

All medications administered orally with 240 mL of water to 12 healthy male human volunteers under fasting conditions.

### Example 7

### Effect of Curing on Pramipexole CR Beads

A series of curing experiments were conducted at various temperatures to obtain a stable formulation. The dissolution results of the cured—CR formulation is shown in Table 4.

TABLE 4
Pramipexole CR Beads - Cured: Dissolution Stability Profile
Dissolution Conditions: USP paddle method 100 rpm, 500 ml
simulated gastric fluid without enzyme at 37° C. pH1.2.
% Pramipexole HCl Released (cured at 60° C./6 hours)

| | | Stored at |
|---|---|---|
| | | 30° C./60% RH for 3 |
| Time (Hours) | Initial | months |
| 1 | 12 | 13 |
| 2 | 14 | 14 |
| 4 | 16 | 16 |
| 6 | 20 | 20 |
| 8 | 29 | 27 |
| 10 | 40 | 37 |
| 12 | 50 | 46 |
| 16 | 67 | 63 |
| 18 | 74 | 71 |
| 20 | 80 | 78 |
| 24 | 86 | 85 |

An optimized curing time of 6 hours at 60° C. was thus identified. This produced a stable product with little change in dissolution profile on storage.

### Example 8

### Pramipexole Bead with Top IR Coat Added

Pramipexole CR beads were coated with a top IR coat of pramipexole. This permits the release of some amount of drug (20-25%) within 2 hours, thus eliminating the lag period. The amount of controlled release polymer was optimized to 27% to obtain the desired Cmax level. The bead was cured at 60° C. for 6 h in a fluid bed dryer to obtain a CR product with little change in dissolution profile on storage.

### Example 9

### Pharmacokinetic Study

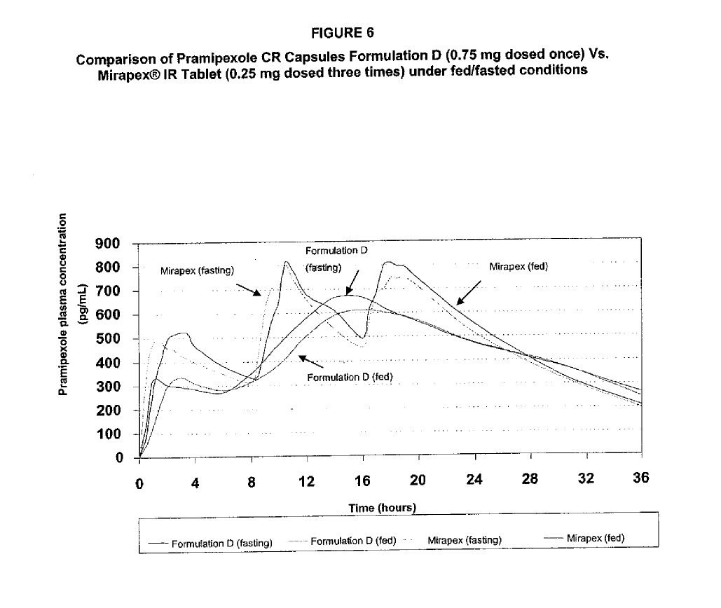

A four-way pharmacokinetic study was performed using pramipexole CR capsules (0.75 mg—23% IR and 77% CR) contain 27% CR polymer (Formulation D) under fed and fasted condition against Mirapex® 0.25 mg (IR Pramipexole tablets). As desired and predicted, in vivo results showed no lag time for the CR capsules and a Cmax lower than the IR tablet. FIG. 6 shows the PK results of the fed/fasted study with a summary of the pharmacokinetic results.

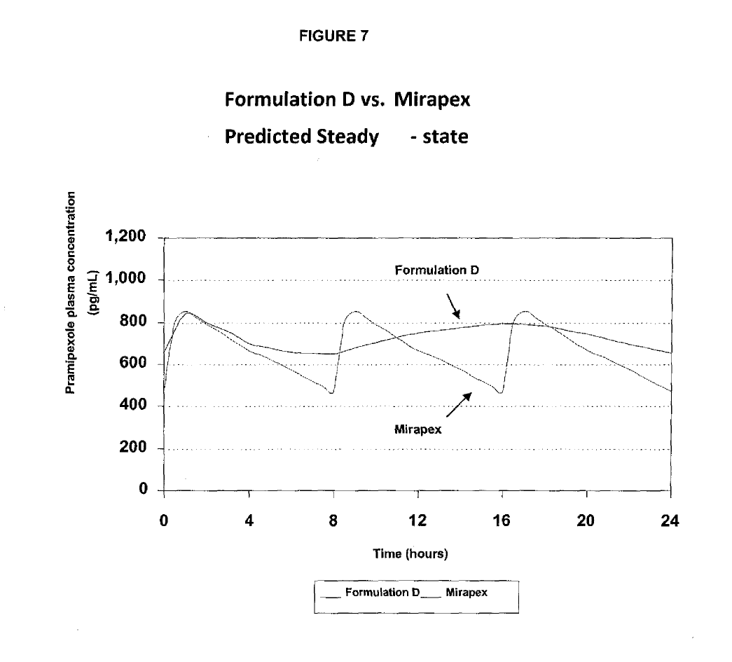

As shown in FIG. 7 (predicted steady-state pharmacokinetics), the CR Formulation D has a much lower fluctuation (lower Cmax/Cmin ratio) than the IR product.

### Example 10

### Stability Study

As shown in Table 5, a study on cured sugar spheres indicated that pramipexole CR showed no changes in the dissolution profile. However, accelerated stability conditions (25° C. and 30° C.) showed a reduction in potency.

TABLE 5
Assay Trend at different storage conditions

Potency - Pamiprexole HCl capsule
(mg) (% label claim)

| | Storage Condition | Storage Condition |
|---|---|---|
| Time Point | 25° C./60% RH | 30° C./65% RH |
| Initial | 0.76 (101.3) | 0.76 (101.3) |
| 3 month | 0.74 (98.67%) | 0.75 (100%) |
| 6 month | 0.71 (94.67%) | 0.69 (92.0%) |

This study shows a significant reduction in the assay value. Thus, pramipexole is very susceptible to heat and moisture. The use of an aqueous moisture barrier coat is imperative for protecting the beads from moisture. The moisture barrier coat has an extremely low moisture transmission rate and provides film-coating protection from environmental moisture. Therefore, a moisture barrier coat at the final stage of manufacturing was added to overcome the effect of moisture on the pramipexole CR formulation.

### Example 11

### Effect of Microcrystalline Cellulose Beads

Though curing and the use of a moisture barrier coat stabilized the assay and dissolution profile, an increase in impurity profile was observed (up to 0.7% increase) when sugar beads were used as substrates. Further investigation revealed that sugar bead and Pramipexole do interact and produce a significant amount of degradant. It was decided to use microcrystalline cellulose (MCC) beads instead of sugar beads since MCC beads are believed to be more inert and should show lesser impurity profiles.

Table 6 shows the results of the stability study.

TABLE 6
Comparison of stability of pramipexole on MCC and sugar beads

| | % Degradation product observed | |
|---|---|---|
| | | Sugar bead |
| Time | MCC bead based formula | based formula |
| Initial | 0.15 | 0.15 |
| 3 month 25° C./60% HR | 0.17 | 0.69 |
| 3 month 30° C./65% HR | 0.18 | 0.86 |

### Example 12

### Fed/Fasting PK Study

A study was carried out on three formulations based on sugar and MCC beads for a fed/fasted PK study. These formulations were:

1) Pramipexole CR beads (sugar spheres) containing an IR component (Formulation D); under fasted conditions.

2) Pramipexole CR beads (MCC spheres) containing no IR component (Formulation E); under fasted conditions.

3) Pramipexole CR beads (MCC spheres) containing IR and CR components (Formulation F); under fasted conditions.

4) Pramipexole CR beads (MCC Spheres) containing IR and CR components (Formulation F); under fed conditions.

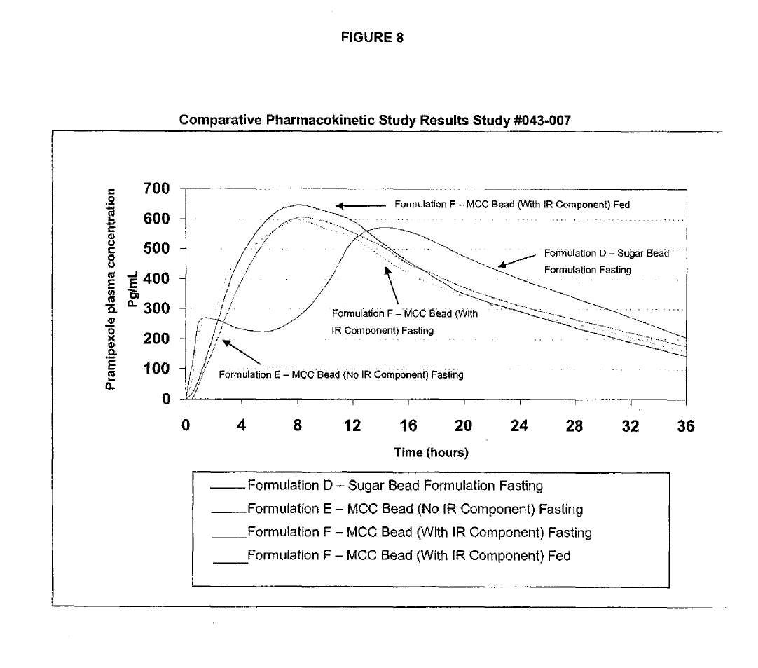

As illustrated in FIG. 8, the PK study showed an excellent plasma profile for a once-daily Pramipexole CR for all 3 prototype formulations. It was also noted that food had no significant effect. Based on all these studies MCC spheres with an IR+CR combination was chosen as the preferred formulation.

Table 7 shows the PK parameters for Formulations D (fasting), D (fed), Mirapex (fasted) and Mirapex (fed). Table 8 shows the dissolution results for Formulations D, E and F. Table 9 shows the PK parameters for Formulations D (fasted), E (fasted), F (fasted) and F (fed).

TABLE 7
Study 043-002 PK Parameters

| Parameters | | Mean | SD | CV (%) | Mean | SD | CV (%) |
|---|---|---|---|---|---|---|---|
| | | Test Formulation D Fasting | | | Formulation D Fed | | |
| AUC₀₋ₜ | (pg · h/mL) | 15582.33 | 2535.89 | 16.27 | 14611.79 | 1487.00 | 10.18 |
| AUC_{0-inf} | (pg · h/mL) | 23285.22 | 8292.03 | 35.61 | 21904.66 | 7454.75 | 34.03 |
| Cₘₐₓ | (pg/mL) | 687.20 | 143.43 | 20.87 | 627.90 | 82.38 | 13.12 |
| Tₘₐₓ | (h) | 15.3 | 1.3 | 8.50 | 16.2 | 1.3 | 8.24 |
| Kₑₗ | (h⁻¹) | 0.0491 | 0.0174 | 35.43 | 0.0486 | 0.0152 | 31.39 |
| T_{1/2 el} | (h) | 17.24 | 10.62 | 61.57 | 16.19 | 7.63 | 47.10 |
| | | Reference (Mirapex) Fasting | | | Reference (Mirapex) Fed | | |
| AUC₀₋ₜ | (pg · h/mL) | 17035.49 | 2135.28 | 12.53 | 17703.03 | 1764.72 | 9.97 |
| AUC_{0-inf} | (pg · h/mL) | 19470.33 | 2649.30 | 13.61 | 20344.78 | 2636.98 | 12.69 |
| Cₘₐₓ | (pg/mL) | 875.92 | 113.33 | 12.94 | 921.47 | 58.65 | 6.36 |
| Tₘₐₓ | (h) | 14.7 | 4.3 | 29.35 | 15.0 | 3.5 | 23.27 |
| Kₑₗ | (h⁻¹) | 0.0825 | 0.0103 | 12.51 | 0.0825 | 0.0123 | 14.96 |
| T_{1/2 el} | (h) | 8.53 | 1.09 | 12.73 | 8.57 | 1.30 | 15.13 |

TABLE 8
Dissolution Results: Formulation D, E and F.

| | | | Formulation | Formulation |
|---|---|---|---|---|
| Time | | Formulation D | E | F |
| (hours) | Tests | % Released | % Released | % Released |
| 1 | Dissolution: | 21 | 9 | 20 |
| 2 | USP paddle | 21 | 20 | 30 |
| 4 | method, 100 rpm | 22 | 38 | 46 |
| 6 | at 37° C. | 26 | 52 | 58 |
| 8 | Simulated Gastric | 36 | 63 | 67 |
| 10 | Fluid, pH = 1.2 | 48 | 69 | 73 |
| 12 | HPLC/UV at | 60 | 75 | 77 |
| 16 | 260 nm | 77 | 83 | 84 |
| 18 | | 82 | 86 | 87 |
| 20 | | 86 | 89 | 89 |
| 24 | | 92 | 93 | 92 |

TABLE 9
Study 043-007 PK Parameters

| Parameters | | Mean | SD | CV (%) | Mean | SD | CV (%) |
|---|---|---|---|---|---|---|---|
| | | Formulation D (Fasted) | | | Formulation E (Fasted) | | |
| AUC₀₋ₜ | (pg · h/mL) | 13083.51 | 2101.21 | 16.06 | 13080.64 | 2749.08 | 21.02 |
| AUC_{0-inf} | (pg · h/mL) | 18569.76 | 7471.45 | 40.23 | 17489.70 | 5390.20 | 30.82 |
| Cₘₐₓ | (pg/mL) | 586.34 | 95.36 | 16.26 | 622.71 | 99.15 | 15.92 |
| Tₘₐₓ | (h) | 15.0 | 1.6 | 10.61 | 8.84 | 1.58 | 17.88 |
| Kₑₗ | (h⁻¹) | 0.0567 | 0.0214 | 37.70 | 0.0495 | 0.0168 | 33.89 |
| T_{1/2 el} | (h) | 14.78 | 8.55 | 57.83 | 15.43 | 4.87 | 31.55 |
| | | Formulation F (Fasted) | | | Formulation F (Fed) | | |
| AUC₀₋ₜ | (pg · h/mL) | 12983.51 | 2371.54 | 18.27 | 13288.61 | 2560.26 | 19.27 |
| AUC_{0-inf} | (pg · h/mL) | 16765.58 | 4061.04 | 24.22 | 16142.42 | 3851.02 | 23.86 |
| Cₘₐₓ | (pg/mL) | 602.41 | 79.12 | 13.13 | 656.53 | 81.35 | 12.39 |
| Tₘₐₓ | (h) | 8.51 | 2.11 | 24.78 | 8.67 | 1.97 | 22.70 |
| Kₑₗ | (h⁻¹) | 0.0495 | 0.0136 | 27.57 | 0.0583 | 0.0150 | 25.79 |
| T_{1/2 el} | (h) | 15.02 | 4.15 | 27.64 | 12.63 | 3.21 | 25.41 |

The above-described embodiments of the present invention are intended to be examples only. Alterations, modifications and variations may be effected to the particular embodiments by those of skill in the art without departing from the scope of the invention, which is defined solely by the claims appended hereto.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. An oral once-daily pramipexole formulation, comprising an immediate-release component and a controlled-release component,
the immediate-release component and controlled-release component comprising pramipexole,
the immediate-release component and controlled-release component forming outer and inner layers on a coated bead respectively, the controlled release component being cured.

16. The formulation of claim 15, further comprising a moisture barrier coating on the immediate release layer.

17. The formulation of claim 15, further comprising one or more excipients.

18. The formulation of claim 15, wherein at least 20% of the dosage of pramipexole is released in vitro within 2 hours after administration, and at least 40% of the dosage of pramipexole is released in vitro within 4 hours after administration.

19. The formulation of claim 15, in a form for admixture with food for administering to a subject in need thereof.

20. A method of producing an oral once-daily pramipexole formulation having both immediate-release and controlled-release components, comprising the steps of:
a) applying a pramipexole solution to coat a pharmaceutically-acceptable bead;
b) applying a coating to the pramipexole-coated bead to produce a bead having a controlled-release layer;
c) applying a film coating to the bead produced in step b);
d) curing the bead produced in step c) to form the controlled release component; and
e) applying a second pramipexole solution to coat the controlled release component produced in step d) to produce a bead having the controlled-release component and an immediate-release component.

21. The method of claim 20, further comprising the step of:
f) applying a moisture barrier coat on the immediate release component.

22. A method of treating or preventing a disease in a subject in need thereof, comprising administering the pramipexole formulation of claim 1 to said subject.

23. The method of claim 22, wherein the disease is associated with a dopamine receptor.

24. The method of claim 22, wherein the disease is Parkinson's Disease or Restless Leg Syndrome.
